(19) United States
(12) Patent Application Publication
(10) Pub. No.: US 2007/0279589 A1
(43) Pub. Date: Dec. 6, 2007

(54) APPARATUS FOR CONTACTLESS EXAMINATION OF AN EYE

(75) Inventors: Norbert Dahmen, Tonisvorst (DE); Georg Langstrof, Haan (DE)

Correspondence Address: SEED INTELLECTUAL PROPERTY LAW GROUP PLLC, 701 FIFTH AVE, SUITE 5400, SEATTLE, WA, 98104, US

(73) Assignee: RHINE-TEC Gesellschaft fur virtuelle instrumentierung mbH, Krefeld (DE)

(21) Appl. No.: 11/750,909

(22) Filed: May 18, 2007

(30) Foreign Application Priority Data
May 18, 2006 (DE) 20 2006 008 017.1
May 24, 2006 (DE) 10 2006 024 473.7

(51) Int. Cl. A61B 3/14

(52) U.S. Cl. 351/206

(57) ABSTRACT

Precise and informative analysis of the corneal endothelium is appropriate for pre-operative diagnostic and for post-operative care of patients, in particular for cataract operations. Analysis of the corneal endothelium is also desirable for the prophylactic support accompaniment of patients in regard to contact lens-induced corneal endothelium damage. The invention provides an image recording device which is mechanically coupled to a lighting device and with which cells of the corneal endothelium which are lit by the lighting device are detected and are visibly represented with an image reproduction device connected to the image recording device.

### CROSS-REFERENCE TO RELATED APPLICATION

This application claims priority to German Application Numbers 20 2006 008 017.1, filed May 18, 2006, and 10 2006 024 473.7, filed May 24, 2006, which applications are incorporated herein by reference in their entirety.

### BACKGROUND

1. Technical Field

The invention concerns an apparatus for contactless examination of an eye, in particular a human eye.

2. Description of the Related Art

Precise and informative analysis of the corneal endothelium is appropriate for pre-operative diagnostic and for post-operative care of patients, in particular for cataract operations. Analysis of the corneal endothelium is also desirable for the prophylactic support accompaniment of patients in regard to contact lens-induced corneal endothelium damage.

### BRIEF SUMMARY

The object of the invention is to provide an apparatus for contactless examination of corneal endothelium of an eye, which can be used in as simple and patient-friendly fashion as possible.

That object is attained by the features of claim 1.

The invention provides an image recording device which is mechanically coupled to a lighting device, in particular a slit lamp, and with which cells of the corneal endothelium which are lit by the lighting device are detected and are visibly represented with an image reproduction device connected to the image recording device.

The image recording device which is preferably in the form of an additional unit or attachment operates in a completely contactless fashion and does not require any probes which have to be brought into contact with the surface of the eye. Risks of infection as well as physical injury during the examination are thus excluded. There is no need whatsoever for local anesthesia.

For the examination process, the lighting device with a fixedly set direction in respect of the lighting beam (slit lamp beam) and the image recording device with a fixedly set viewing direction or image recording direction can be moved jointly on a base member. The image recording device preferably includes a video camera with a camera objective. An image evaluation device and the image reproduction device are connected to the video camera.

The image evaluation device can have an image selection device which, upon movement of the lighting device and the image recording device, sorts successively recorded video images of the corneal endothelium cells in accordance with their image quality, in which respect more than three video images are evaluated. The image evaluation device can have a storage means for storing the video images entailing good image quality, in particular with sharply reproduced corneal endothelium cells. The image evaluation device can ascertain the cell density within a given image region of the video image, by means of a computer. The geometrical configuration of the image region within which analysis, in particular cell density, is to be ascertained, can be established by the operator.

The cell density distribution of cell surfaces and cell shapes can be displayed on the image reproduction unit within a few seconds for assessment and possibly for post-processing. The standardized mode of operation of the system ensures results which can be reproduced at any time. Besides the video image recordings the analysis results can be represented both in numerical terms and also in graphic form. The corresponding data and video images can be stored and can be subsequently checked at any time. Comparison with earlier examinations is also thus possible.

The device may be used also for producing images of the illuminated sections generated by the slit lamp of the cornea and of the lachrymal fluid on the surface of the cornea. The thickness of the cornea and/or the thickness of the lachrymal fluid or of the lachrymal film on the surface of the cornea can be evaluated by the image evaluation device from the video images. Further, the velocity of the lachrymal film can be determined by the analysis of the successively produced video images. A determination of the viscosity of the lachrymal third fluid may be deducted on the basis of the velocity of the lachrymal film on the cornea. Additionally, breaks of the lachrymal film can be recognized from the images of the lachrymal film.

The invention further concerns an apparatus for positioning an objective of the image recording device, with which eye components which are lit by the lighting device of the slit lamp, in particular cells of a corneal endothelium of an eye, are to be detected.

That provides in a simple fashion for exact orientation and positioning of the objective of the image recording device with respect to the lighting device, in particular with respect to the slit lamp.

For that purpose, a fixing device can be provided at the image recording device, for example by means of a holding plate, which fixing device can be fitted into a bore oriented in relation to the pivot axis of the lighting device, preferably on the carrier of the slit lamp, for a pivotal movement of the image recording device about the pivot axis. That provides that the image recording device and the objective of the image recording device can be pivoted about a common pivot axis with the lighting device, in particular the slit lamp. For that purpose the fixing device can have a spreadable sleeve which projects into the bore on the slit lamp holder. By means of a screw connection projecting through the cavity of the sleeve, the sleeve can be spread radially outwardly so that the objective of the image recording device and the image recording device can be fixed at a given angle with respect to the lighting device of the slit lamp unit. When the sleeve is spread radially outwardly a positively locking frictional engagement is formed between the sleeve and the inside wall of the bore. When the screw connection is loosened or released the lighting device and the image recording device can be pivoted into new angular positions about the common pivot axis.

For the purposes of fixing the spreadable sleeve, the axial prestressing which is exerted with the screw connection that projects through the sleeve, on the two parts of the screw connection, is utilized to increase the outside diameter of the sleeve and thus to provide the positively locking frictional engagement with the inside wall of the bore. For that purpose one of the screw connection parts, preferably a nut into which the thread of screw shank engages, can have a spreading cone which is fitted into a correspondingly shaped spreading cone on the sleeve. By virtue of the axial prestressing afforded by the screw connection, the sleeve is then spread radially outwardly. Preferably the sleeve and the nut are arranged non-rotatably relative to each other in the bore.

That provides a fixing device with which the optical axis or viewing direction of the objective of the image recording device and the direction of the lighting beam of the lighting device are held non-rotatably in a given angle relative to each other in the common movement during the eye examination procedure. That fixing device can also be implemented with other structural components. For that purpose for example a pin fixed to the image recording device can be non-rotatably fitted into the bore on the slit lamp unit. For that purpose the bore and the pin can be of cross-sectional shapes which differ from a round cross-section so that the non-rotational connection therebetween is ensured. Furthermore the pin can be fixedly connected to a mechanical rotation-preventing means which comes into non-rotational engagement with the outside contour surrounding the bore on the slit lamp, for example on the pivot base or holder of the slit lamp.

The described fixing devices provide that the image recording device can be connected if required to a conventional slit lamp unit. It is however also possible for the slit lamp and the image recording device to be provided in one unit in which they can be moved on a base member jointly non-rotatably in a given angular position with respect to each other in the eye examination.

The objective can have an objective tube in which the objective lens system is disposed. The objective lens system can be adjusted in the direction of its optical axis by rotation of the objective tube with respect to a holder provided on the image recording device. It is possible in that way to provide for sharp focusing of the eye component of which the image is to be formed, in particular the corneal endothelium cells which are to be imaged on an enlarged scale, for image detection, for example by means of a video camera.

### BRIEF DESCRIPTION OF THE SEVERAL VIEWS OF THE DRAWINGS

The invention will be described in greater detail hereinafter by means of embodiments by way of example with reference to the Figures in which:

FIG. 1 shows an embodiment of the invention,

FIG. 2 shows a plan view of components of the embodiment of FIG. 1,

FIG. 3 shows a portion of a video image of corneal endothelium cells, which is produced with the embodiment,

FIG. 4 shows an adjustment device for the embodiment,

FIG. 5 shows a block circuit diagram illustrating essential functional elements of the embodiment,

FIG. 6 shows an embodiment of a positioning device with which the lighting device is arranged on the slit lamp unit,

FIG. 7 diagrammatically shows the arrangement of an objective which is provided in the image recording device in the embodiment,

FIG. 8 shows a sectional view taken along section line A-A in FIG. 7 through an object holder,

FIG. 9 shows a perspective view of a further embodiment of the positioning device,

FIG. 10 shows a perspective view of yet a further embodiment of the positioning device, and

FIG. 11 shows a fixing device which can be used in the further embodiments of FIGS. 9 and 10.

### DETAILED DESCRIPTION

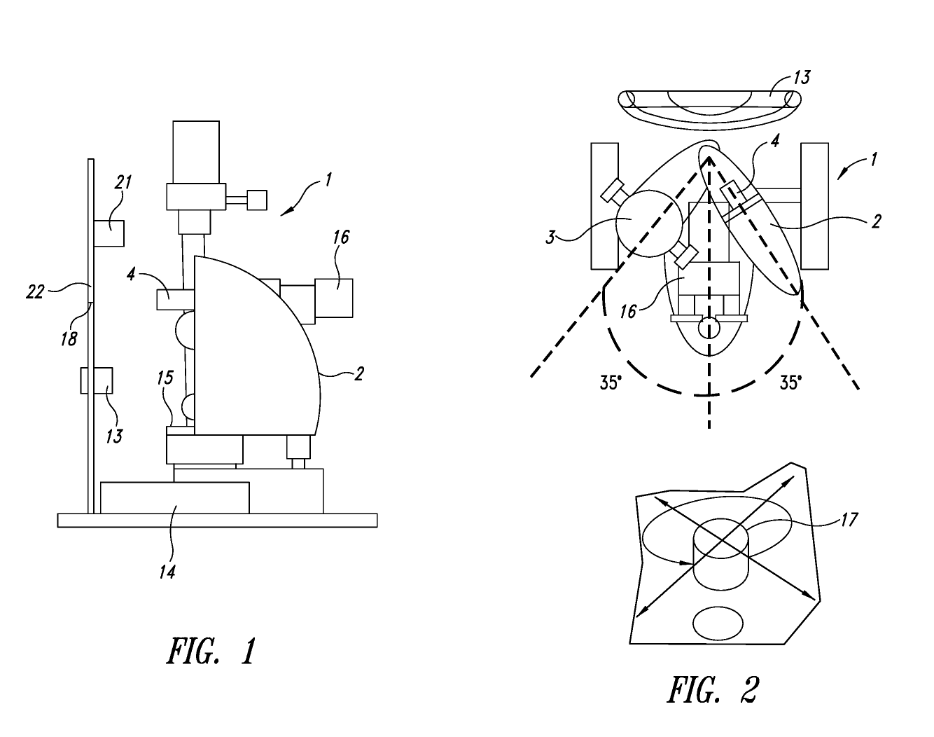

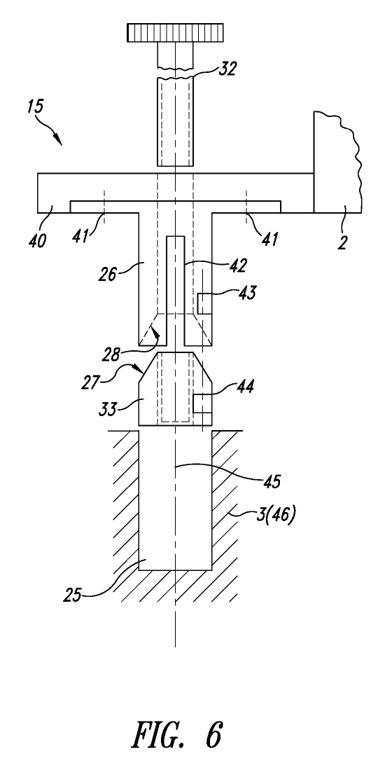

The embodiment illustrated in FIGS. 1 and 2 includes a slit lamp 1 which can be of a conventional design configuration as well as an image recording device 2. The image recording device 2 and the slit lamp unit 1 are mechanically coupled in such a way that corneal endothelium cells which are lit by means of a lighting device 3 of the slit lamp 1 can be detected. The corneal endothelium cells are visibly represented by means of an image reproduction device 6 (see FIG. 5) connected to the image recording device 2. For that purpose, in a housing the image recording device 2 has a video camera 20 with an adjustable camera objective 4. A suitable image recording device 6 is a screen of a laptop or a PC apparatus or also a suitable monitor. The lighting device 3 of the slit lamp produces a narrow light pencil which in known manner produces an optical section through the transparent eye tissue in particular of the cornea of the eye. In the illustrated embodiment the light pencil produced by the lighting device 3 can have for example a slit width of about 1 mm and a slit height of about 5 mm.

The image recording device 2 which is in the form of an additional unit or attachment in relation to the slit lamp 1 can be mechanically coupled to the slit lamp unit 1 with a fixing device 15 which is inserted into a central bore in the slit lamp unit until reaching a condition of abutment and is locked there.

The mechanical connection to the slit lamp unit 1 is preferably such that the lighting device 3 of the slit lamp unit 1 with a fixedly set direction of the lighting beam formed by the narrow light pencil from the slit lamp and the image recording device 2 with a fixedly set viewing direction or image recording direction can be moved jointly on a base member 14 of the slit lamp unit 1.

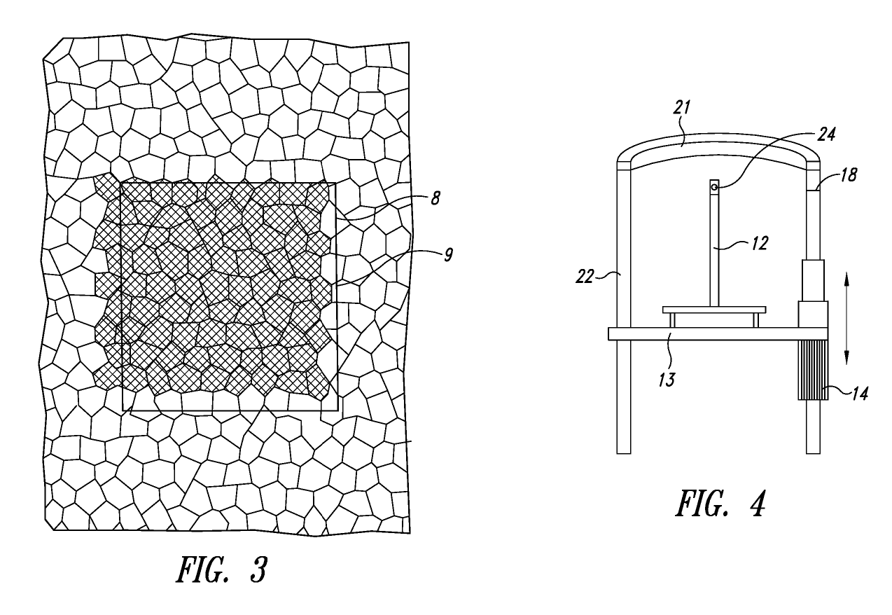

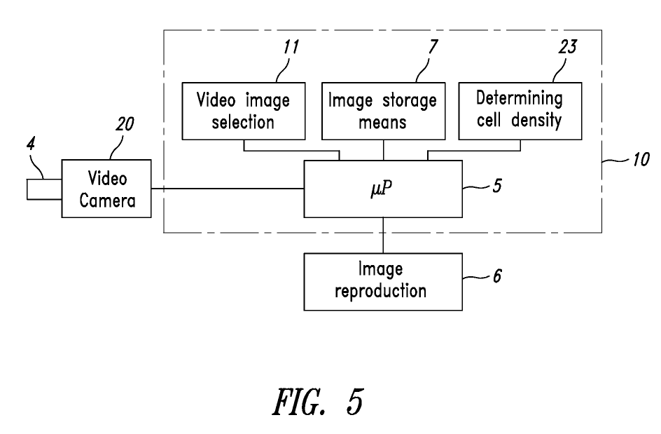

For that purpose adjustment of the apparatus can preferably be implemented to start with. In that case an adjustment accessory 12 is placed on a chin support 13 (see FIG. 4). At its upper end the adjustment accessory 12 which is in the form of a bar has a reticule 24 or an image of a reticule. It is also possible to provide another form which approximately corresponds to the cell pattern of the corneal endothelium cells to be recorded, at the upper end of the adjustment device 12 in bar form. As shown in FIG. 2 the beam direction of the lighting device 3 is set at an angle of about 35° with respect to the axis of a binocular microscope 16 at the slit lamp 1. Likewise the viewing direction or the optical axis of the camera objective 4 is also set at an angle of about 35° with respect to the axis of the microscope 16. The lighting direction and the optical axis of the camera objective 4 or the viewing direction of the recording device 2 thus include an angle of about 70° (see FIG. 2). The slit for the light pencil from the slit lamp is set to be as narrow as possible. By means of an adjusting device 17, for example a joystick, which can control the joint movement of the lighting device 3 and the image recording device 2 in one plane in all directions of movement, the lighting device 3 and the image recording device 2 are moved in such a way that the image of the light pencil of the slit lamp is produced approximately centrally on the reticule 24. With a fixed arrangement, the adjustable camera objective 4 is set in such a way that the video image of the reticule appears on the image reproduction device 6, for example the monitor screen or the like, centrally and with the sharpest possible outlines.

So that the reticule 24 is at the height of the optical axis of the camera objective the chin support 13 can be adjusted by means of an adjusting screw or another adjusting means having the same effect, so that the reticule 24 is moved to the height of a marking 18 provided on a frame 22 of the chin support 13.

When examining the eye of a patient, the chin of the patient is rested on the chin support 13 and the forehead of the patient bears firmly against a head support 21 on the frame 22. The eye of the patient to be examined is adjusted to the height of the marking 18 and thus to the height of the optical axis of the camera objective 4 by means of an adjusting screw 19.

In the recording mode during the examination the optical section which the light pencil from the slit lamp produces at the cornea of the patient is set sharply and centrally on the cornea. The position of the optical section can vary slightly depending on the respective viewing direction of the patient. A signal and in particular an optical signal can possibly be produced when the central position is reached by the image evaluation device. Fine adjustment of the jointly moved arrangement consisting of the image recording device 2 and the lighting device 3 can be implemented by means of the adjusting device 17, until the image of the corneal endothelium cells is produced centrally and in sharp focus on the reproduction device 6. During that controlled movement of the image recording device 2 and the lighting device 3 successive endothelium images are produced by the video camera 20 which is provided in the image recording device 2, and those images are rendered visible at the image reproduction device 6. More than three video images are evaluated, in which case for example up to 50 successive video images can be produced and evaluated. Images involving a good reproduction quality are put into intermediate storage in an image storage means 7 of an image evaluation device 10 (see FIG. 5). Selection of the images entailing a good reproduction quality can be effected by means of an image selection device 11 of the image evaluation device 10. The examination of the eye of the patient can be concluded when evaluatable endothelium images are available and were visible at the image reproduction device 6. A suitable video image can be selected from the video images put into intermediate storage in the image storage means 7, and rendered visible at the reproduction device 6. FIG. 3 diagrammatically shows an image portion of the corneal endothelium cells reproduced on the image reproduction device 6.

For the evaluation procedure a given image region 8 which is established by boundary lines on the reproduction device 6 can be selected for cell density analysis, as is shown in FIG. 3. The endothelium cells in the image region 8 can be provided with a marking 9. This can involve an area color marking or also a color marking in point form, in respect of the individual cells of which the image is shown within the given image region 8. Correction of the color marking and also correction of the respective positions of the boundary lines which surround the given image region 8 can possibly be implemented by means of mouse clicking. The given image region 8 can be of a rectangular, square or other suitable geometrical configuration. Computer-aided detection, for example counting, of the preferably marked cells present in the given image region 8 makes it possible to determine the cell density in the evaluation device 10 by means of a computer 5 of the evaluation device 10, having regard to the content in relation to surface area of the given image region 8 (determining cell density as indicated at 23 in FIG. 5).

For that purpose, as can be seen from FIG. 5, the evaluation device 10 is connected to the video camera 20 provided in the image recording device 2 and evaluates the digitized electrical video signals supplied by the video camera, for reproduction at the image reproduction device 6.

The image evaluation device 10 can be disposed in the housing of the image recording device 2. It is however also possible for the functions of the evaluation device 10 to be stored on a transportable storage means, for example a diskette, in the form of software, and installed in the computer associated with the reproduction device 6, when the respective examination is carried out. That analysis software can provide for control of the respective live video image of the corneal endothelium, a central measurement point of the corneal endothelium, and automatic image selection in respect of representation of the cell density, the cell surfaces and the cell morphology. That also provides for quick and simple analysis of the corneal endothelium.

So that images entailing good image reproduction quality are achieved, it is advantageous if the lighting device of the slit lamp 1 and the objective 4 of the image recording device 2 are set exactly relative to each other. That purpose is served by a positioning device which ensures that the image recording device 2 and its objective 4 as well as the lighting device 3 are pivoted about a common pivot axis 45. The embodiment illustrated in FIG. 6 shows details of the fixing device 15 with which that requirement is met. For that purpose, a bore 25 of circular diameter is provided on the slit lamp device, in particular at the pivot base of the lighting device 3, about the pivot axis 45. Fitted into that bore 25 is a nut 33 which is of a conical configuration at its upwardly facing surface, thereby providing a spreading cone 27 with an upwardly directed conical annular surface. A sleeve 26 is fitted on to the spreading cone 27. The underside of the sleeve 26 also has a spreading cone 28 with a substantially annular conical surface which is matched to the spreading cone 27. The sleeve has slots 42 which extend in parallel relationship with the axis thereof so that radially outwardly directed deformation of the peripheral portion of the sleeve can be effected. The sleeve is also disposed in the bore 25 when it is fitted on to the spreading cone of the nut 33.

The sleeve 26 has a flange which is fixed with screw connections 41 to the underside of a holding plate 40. The holding plate 40 is fixed to the housing of the image recording device 2.

A screw 32 can be passed through a bore in the holding plate 40 and the cavity in the sleeve 26 and screwed with the thread on the screw shank to the nut 33. When the screw connection afforded in that way is tightened, an axial prestressing force is produced, by which the two spreading cones 27 and 28 are pressed against each other. When the screw connection is tightened, the nut 33 and the sleeve 26 can be held non-rotatably relative to each other by a pin being arranged in bores 43 and 44 in the sleeve 26 and in the nut 33. The spreadability of the sleeve 26 provides that the peripheral surface of the sleeve 26 is pressed against the inside wall of the bore 25 so that frictional engagement provides a fixing action both in the axial direction and also in the rotary direction. Using simple means, that provides for positioning of the image recording device 2 on the slit lamp unit 1 and in particular on the lighting device 3.

For greater ease of actuation of the screw connection, the screw 32 can be in the form of a knurled-head screw which is secured in the released condition for example by a circlip with respect to the holding plate 40 so that the screw 32 is prevented from falling out, even when the screw connection is released or loosened. When the screw connection is released or loosened the image recording device 2 can be pivoted about the pivot axis 45 which can form a common pivot axis with the lighting device 3, and can be moved into a desired angular position. That desired angular positioning is maintained in the eye examination procedure, by the above-discussed fixing arrangement.

That provides that the optical axis 35 of the objective 4 is directed on to the section produced by the light pencil from the slit lamp at the cornea of the patient. The objective 4 has an objective tube 30 which is screwed with a male screwthread 36 into a female screwthread 34 of an objective holder 31. The objective holder 31 is fixed to the inside of the housing of the lighting device 3. The objective tube 30 projects beyond the front surface of the housing of the image recording device 2, as can be seen from the Figures. Arranged in the objective tube 30 is an objective lens system 29 which, upon rotation of the objective tube 30, is displaced axially in the direction of the optical objective axis 35.

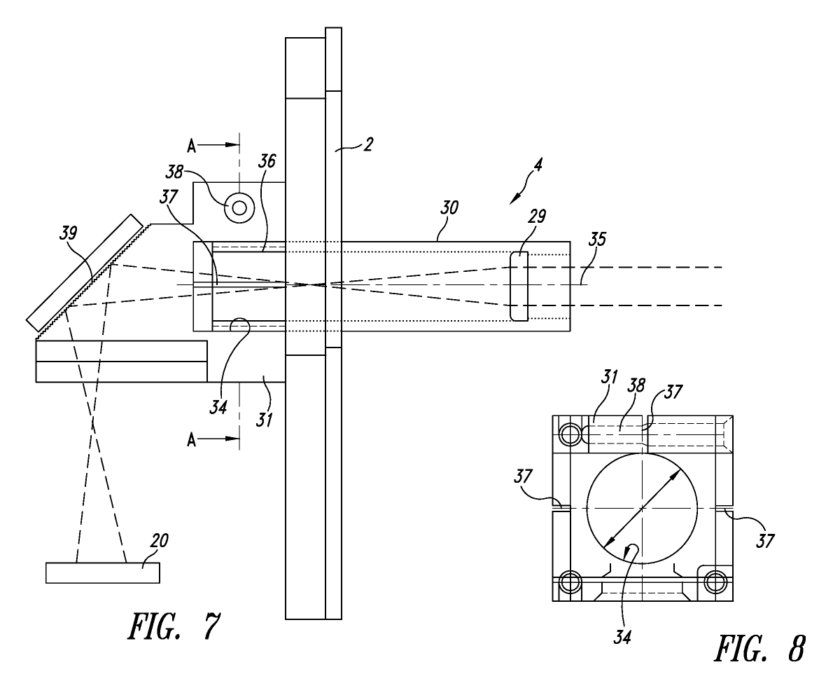

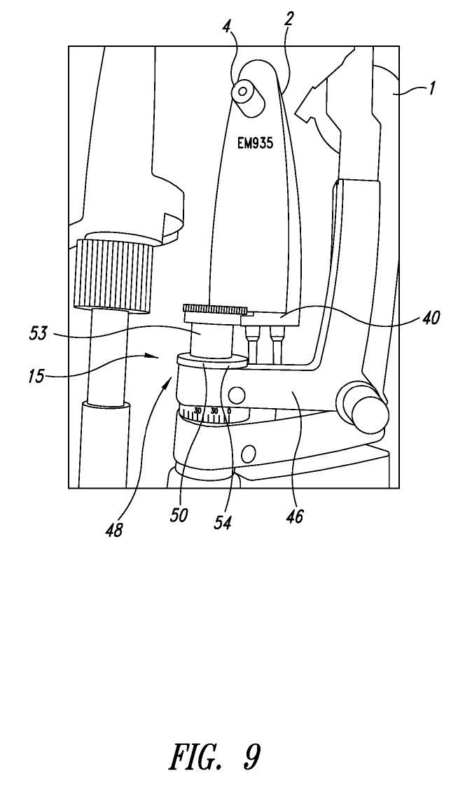

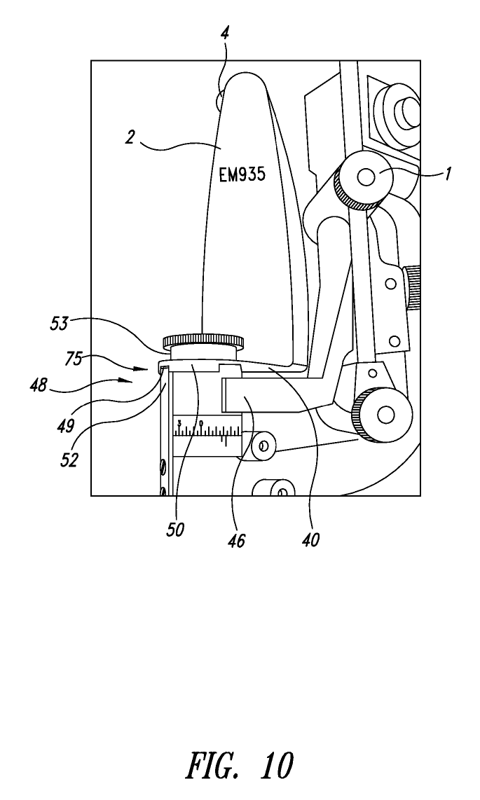

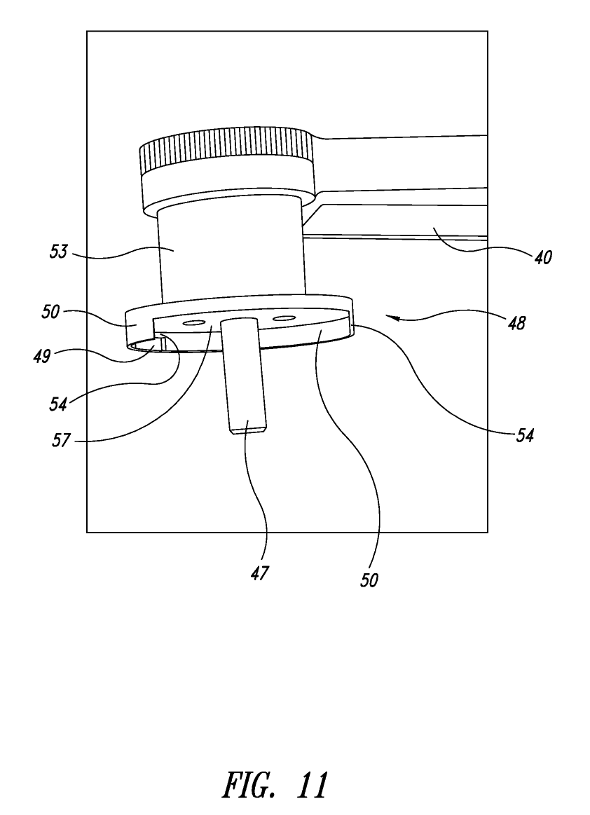

The ease of the rotary movement and therewith the axial displaceability of the objective 4 can be adjusted. For that purpose there are then provided on the objective holder 31 one or more slots 37 which extend in parallel relationship with the optical objective axis 35. The diameter D (see FIG. 8) of the female screwthread 34 on the objective holder 31 can be altered by means of a clamping screw 38 which extends perpendicularly to the optical objective axis 35. That alteration can be between easy movement and a clamping fit of the objective 4 in the objective holder 31. The material of the objective holder 31 is suitably deformable for that purpose. It is possible in that way to fix the set focal length and possibly enlargement of the image to be detected, in the optical section of the light pencil of the slit lamp. The beam path of the objective 4 is directed on to the video camera and here in particular on to the light-sensitive surface thereof in the image recording device 2 by way of a deflection mirror 39 which is arranged fixedly with respect to the housing. The video camera 20 can be for example in the form of a CCD camera. As already described an image reproduction device which is not shown in greater detail is connected to the video camera 20. FIGS. 9 to 11 show further embodiments for non-rotatably connecting the image recording device 2 to the slit lamp 1. The fixing device 15 provided in these embodiments has a pin 47 (see FIG. 11) which is fixed to the image recording device 2 and which can be inserted into the bore 25 provided on the slit lamp unit. In the embodiments in FIGS. 9 and 10 the pin 47 is inserted into the bore 25. The bore 25 is disposed at the end of a holder 46 for the slit lamp 1. That holder 46 in the illustrated embodiments is the pivot base of the slit lamp 1. The pin 47 is fixed to the end of the holding plate 40 and fixedly connected by way of that holding plate 40 to the image recording device 2. Fixing of the pin 47 at the underside of the holding plate 40 can be effected by way of a spacer 53, the height of which is respectively dimensioned according to the configuration of the holder mounting arrangement provided on the holder 46. In the embodiment in FIG. 9 the height of the spacer 53 is greater than in the embodiment in FIG. 10.

Fixed to the underside of the spacer 52 is a plate 51 which at its peripheral edge has a downwardly projecting flange 50. The underside of the plate 51 forms a contact surface which can bear against the upwardly disposed surface of the rotary mounting arrangement for the holder 46 of the slit lamp 1. In this case, in the illustrated embodiments, the downwardly projecting flange 50 partially embraces the outside contour of the rotary mounting arrangement of the holder 46. The particular configuration of the flange 50 in conjunction with the outside contour of the rotary mounting arrangement of the holder 46 provides a rotation-preventing means 48 which provides that, in relation to the fixing device 15, the image recording device is non-rotatably connected to the slit lamp 1. The optical axis of the camera objective 4 and the direction of the lighting beam from the slit lamp 1 are thus non-rotatably connected together.

In the embodiment shown in FIG. 9 the non-rotatable connection is effected by the holder 46 projecting through an opening in the flange 50, the opening being adapted to the width of the holder 46. That opening in the flange 50 is delimited by abutment surfaces 54 which bear laterally against the holder 46. The other parts of the flange 50 project downwardly and bear against the outside contour of the rotary mounting arrangement of the holder 46, as can be seen from FIG. 9.

In the embodiment of FIG. 10, provided on the rotary mounting arrangement of the holder 46 is a projection 52 which projects into an opening 49 in the flange 50. That provides that the image recording device 2 is non-rotatably positioned with respect to the slit lamp.

The embodiments in FIGS. 9 to 11 provide for simple positioning of the image recording device 2 in the region of the rotary mounting arrangement of the slit lamp 1 at its holder 46 or pivot base. The image recording device 2 can be easily achieved in the fixing device 15 by insertion of the pin 47 into the bore 25 at the rotary mounting arrangement. Removal of the image recording device 2 from the fixing device 15 is also an easy operation by virtue of withdrawing the pin 47 from the bore 25.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet, are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. An apparatus for contactless examination of corneal endothelium of an eye, including an image recording device which is mechanically coupled to a slit lamp and with which components of the cornea and/or a lachrymal film on the surface of the cornea, which are lit by a lighting device of the slit lamp, can be detected, and an image reproduction device which is connected to the image recording device and with which the detected components of the cornea and/or of the lachrymal film can be visibly represented.

2. The apparatus according to claim 1 wherein the lighting device of the slit lamp and the viewing direction of the image recording device are fixedly set relative to each other and the image recording device with the slit lamp are movable jointly on a base member.

3. The apparatus according to claim 2 wherein the lighting device and the image recording device are pivotable about a common axis.

4. The apparatus according to claim 1 wherein the image recording device has a video camera with a camera objective, wherein an image evaluation device and the image reproduction device are connected to the video camera.

5. The apparatus according to claim 1 wherein the image evaluation device has an image selection device which sorts video images of the corneal endothelium cells which are successively recorded upon movement of the lighting device and the image recording device in accordance with their image quality.

6. The apparatus according to claim 1 wherein the image evaluation device has an image storage means for storage at least of the video images of good image quality.

7. The apparatus according to claim 1 wherein the image evaluation device has a computer which ascertains the cell density of corneal components, especially of the cornea endothelium and/or the velocity of the lachrymal film on the surface of the cornea and/or the thickness of the cornea and/or the thickness of the lachrymal film particularly within a given image region of the video image.

8. The apparatus according to claim 1 wherein the boundary lines surrounding the image region can be visibly represented on the image reproduction device.

9. The apparatus according to claim 1 wherein the corneal endothelium cells of which the image is formed in the given image region are markable.

10. The apparatus according to claim 1 wherein an adjustment accessory can be positioned on a chin support with a reticule which is lit by the lighting device and which is provided approximately at eye height, wherein for adjustment purposes a sharp video image can be set by means of adjustment of a camera objective.

11. The apparatus according to claim 1 wherein the camera objective is to be arranged with its optical axis approximately at the height of the eye to be examined.

12. The apparatus according to claim 1 wherein the chin support is adjustable in height.

13. The apparatus according to claim 1 wherein the optical axis of the objective of the image recording device and the direction of the lighting beam of the slit lamp are held in a given angular position relative to each other by means of a fixing device.

14. The apparatus according to claim 1 wherein the image recording device and the slit lamp which are non-rotatably connected together by means of the fixing device are mounted on a base plate jointly controlledly movably in all directions of movement.

15. The apparatus according to claim 1 wherein the image recording device and the slit lamp can be connected together in the fixing device by a plug-in connection.

16. The apparatus according to claim 1 wherein the fixing device has a plug-in connection which is in pin form and which can be inserted into a bore in a holder of the slit lamp.

17. The apparatus according to claim 13 wherein the fixing device on the image recording device has a spreadable sleeve which can be inserted into a bore on the slit lamp unit, which bore is aligned in relation to the pivot axis of the lighting device, for a pivotal movement of the image recording device about the pivot axis, and that the sleeve is radially outwardly spreadable by a screw connection which projects through the cavity of the sleeve, for fixing the objective of the image recording device in a given angle relative to the lighting device by positively locking frictional engagement with the inside wall of the bore.

18. The apparatus according to claim 17 wherein a spreading cone is provided at least on a screw connection part of the screw connection, said spreading cone engaging into a corresponding spreading cone on the sleeve.

19. The apparatus according to claim 13 wherein the fixing device has a mechanical rotation-preventing means which non-rotatably engages into the outside contour of the holder of the slit lamp.

20. The apparatus according to claim 19 wherein the rotation-preventing means has a contact surface which can bear against the rotary mounting arrangement of the holder and which is surrounded by a partially peripherally extending flange which can be brought into non-rotary engagement with the outside contour of the rotary mounting arrangement of the holder.

21. The apparatus according to claim 1 wherein the objective lens system arranged in an objective tube is adjustable in the direction of the optical objective axis by rotation of the objective tube with respect to a housing of the image recording device.

22. The apparatus according to claim 21 wherein the ease of the adjusting movement of the objective is adjustable.

23. The apparatus according to claim 21 wherein the objective tube is axially displaceably held with screwthread engagement in an objective holder provided on the image recording device, for axial adjustability of the objective.

24. The apparatus according to claim 21 wherein the screwthread diameter of the female screwthread provided on the objective holder is variable.

25. The apparatus according to claim 21 wherein the objective holder has one or more slots which extend in parallel relationship with the optical objective axis and the slot width of which is variable by varying the screwthread diameter at the objective holder by means of a clamping screw.

26. The apparatus according to claim 21 wherein arranged in the image recording device in the beam path direction after the objective is a deflection mirror which deflects the beam path through the objective towards a video camera.

27. The apparatus according to claim 26 wherein the deflection angle is about 90°.
